(19) United States
(12) Patent Application Publication
(10) Pub. No.: US 2025/0312525 A1
(43) Pub. Date: Oct. 9, 2025

(54) FLUID COLLECTION APPARATUS

(71) Applicant: ConvaTec Limited, Flintshire (GB)

(72) Inventors: Robert Gordon Maurice Selby, Flintshire (GB); Simon John Weddelow, Flintshire (GB); Lawrence Mark Baker, Flintshire (GB)

(21) Appl. No.: 18/975,127

(22) Filed: Dec. 10, 2024

Related U.S. Application Data
(63) Continuation of application No. 16/764,272, filed on May 14, 2020, now Pat. No. 12,161,792.

(30) Foreign Application Priority Data
Nov. 16, 2017 (GB) 1719014.1
Nov. 16, 2017 (GB) 1719027.3

(51) Int. Cl. A61M 1/00

(52) U.S. Cl. CPC A61M 1/784; A61M 1/60; A61M 1/88; A61M 1/98; A61M 2205/7536

(57) ABSTRACT

Fluid collection apparatuses and methods of use in negative pressure therapies are provided.

### CROSS-REFERENCE

This patent application claims the benefit of GB1719014.1 filed Nov. 16, 2017; and GB1719027.3 filed Nov. 16, 2017; each of which is incorporated herein by reference in their entirety.

### BACKGROUND

Exudating wounds may be treated by providing negative pressure to the space above the wound to promote healing in a process often referred to as negative pressure wound therapy (NPWT). During NPWT, effluent such as exudate is removed from the wound and collected. In some therapies, the effluent is stored in a fluid collection apparatus positioned between the source of negative pressure and a the wound site. Typically the apparatus has a specific orientation to prevent exudate drawn into the apparatus from reaching the air outlet of the collection apparatus or otherwise prevent excess exudate from being drawn into the collection apparatus and/or fluid or other undesirable material being drawn into the negative pressure supply. As a consequence of this specific orientation, the patient may be restricted in mobility. In addition, care must be taken so that the specific orientation is not disrupted, which can interrupt the NPWT process.

### BRIEF SUMMARY OF THE INVENTION

In one aspect, disclosed herein is an apparatus configured to allow for orientation independence during negative pressure wound therapy (NPWT). For instance, an apparatus comprising multiple sides (e.g., six sides), may be positioned in such a way that any of the multiple sides may be laid against a horizontal or substantially horizontal surface in use. As a non-limiting example, the apparatus may be capable of operating upside down such as may occur if carried in a bag when mobile or otherwise supported or placed in that orientation. The apparatus may additionally or alternatively be hung to a drip stand or other suitable device or affixed to a wall by an attachment point located on any of the multiple sides. This orientation independence may be achieved, for example, by arranging a first impeding element at a first end of a first fluid pathway defined by a first fluid defining element, such as a chamber or tube, in fluid communication with a source of negative pressure, and a second impeding element at a second end of the first fluid pathway, such that during NPWT, air may be preferentially drawn through the impeding element and into the first fluid pathway while liquid flow is impeded. Preferably, the first end of the first fluid defining element and a first side of the apparatus are in substantially fluid tight communication. Preferably, the second side of the apparatus opposes the first side of the apparatus. In an exemplary embodiment, as the apparatus fills with liquid, liquid may be drawn through the impeding elements and into the fluid pathway. As a non-limiting example, the liquid is drawn through the impeding elements and into the fluid pathway when the apparatus reaches a full or nearly-full state. To hinder liquid from being drawn from the fluid pathway into the source of negative pressure, the fluid pathway may comprise an air permeable member positioned within the fluid pathway to separate liquid drawn into the fluid pathway from the source of negative pressure. In some embodiments, the air permeable member is a filter that impedes liquid and solid material from passing through the member. In some instances, liquid drawn into the fluid pathway may saturate the air permeable member or the air permeable member may reach a predefined saturation level, which may cause an undesired change to the intended negative pressure level. In some cases, this pressure change may be used to detect or sense that the apparatus may be in a full or nearly-full state, and cause generation of negative pressure to halt. Accordingly, liquid may be prevented from entering into the source of negative pressure through the chamber once the air permeable member is saturated or reaches a pre-defined saturation level. In other instances, undesirable material such as wound tissue or absorbent material within the apparatus may block or clog the air permeable member which may cause an undesired change to the intended negative pressure level. In some cases, this pressure change may be used to detect or sense that the apparatus may not be functioning properly, and cause generation of negative pressure to halt. In some cases, this negative pressure change may be due to both of the aforesaid conditions. In some instances, the apparatus comprises an optional aromatic cartridge that may prevent external release of noxious odors drawn into the apparatus. The apparatus may further include one or more optional supports, such as a web or anchor to further retain the impeding element or elements in the chamber.

In some embodiments, disclosed herein are devices for negative pressure wound therapy comprising a collection vessel, a first fluid defining member configured to be in fluid communication with a source of negative pressure, a first impeding element positioned at a first end of the first fluid defining member, a second impeding element positioned at a second end of the first fluid defining member, and a second fluid defining member defining a pathway for dispensing fluid drawn from a wound site of a patient during negative pressure wound therapy into a collection region of the collection vessel; and wherein the first impeding element impedes fluid dispensed within the collection region from entering a first end of the first fluid defining member and the source of negative pressure, and the second impeding element impedes fluid dispensed within the collection region from entering a second end of the first fluid defining member and the source of negative pressure.

In some embodiments, the first and/or second impeding element of the devices disclosed herein comprise foam, in some instances open cell foam, and in further instances the impeding elements comprise polyurethane, polyether, polyvinyl alcohol (PVA), or a combination thereof. In some embodiments, the foam is a reticulated polyurethane foam. In some embodiments, the first fluid defining member is configured to hold the first and the second impeding elements in place within the fluid collection apparatus. In some embodiments, the first fluid defining member has a circular cross-section and/or may comprise a polycarbonate material. In yet other embodiments, the inner diameter of the first fluid defining member is between about 13 and about 23 mm, and the outer diameter of the first fluid defining member is between about 17 mm and about 27 mm.

In yet other embodiments, the devices disclosed herein further comprises a filter positioned within the first fluid defining member. In some embodiments, the filter is a hydrophobic filter; alternatively the filter comprises a pore size of between about 0.2 micron to about 0.8 micron. In other embodiments, the filter comprises polyethersulfone (PES), polytetrafluorethylene (PTFE), cellulose acetate, or a cellulose nitrate membrane.

In yet other embodiments, the devices disclosed herein further comprises a carbon filter. In yet other embodiments, the carbon filter comprises from about 25 g/m2 to about 200 g/m2 of activated carbon.

In some embodiments, the second fluid defining member comprises an elastomer, plastic, polyvinyl chloride (PVC), silicone, ethylene propylene diene monomer (EPDM), Viton, or a combination thereof. In some embodiments, the devices disclosed herein further comprises an absorbent material; in other embodiments, the absorbent material comprises a superabsorbent material, a fibrous structure impregnated with the superabsorbent material, sodium polyacrylate and cellulose pulp in the form of a sheet material, or combinations thereof. In other embodiments, the absorbent material comprises one or more layers of absorbent material within the fluid collection apparatus. In other embodiments, the absorbent material is provided within a sachet. In yet other embodiments, the sachet is dissolvable. In yet other embodiments, the devices disclosed herein further comprises a first wicking layer. In still other embodiments, the first wicking layer is positioned between the first impeding element and the absorbent material. In still other embodiments, the second fluid defining member extends through the first impeding element and comprises an outlet end positioned adjacent to the first wicking layer to wick the dispensed fluid into the absorbent material. In yet other embodiments, the outlet end of the second fluid defining member is positioned within about 10-60 mm of the first wicking layer. In yet other embodiments, the devices disclosed herein further comprises a second wicking layer. In still other embodiments, the second wicking layer is positioned between the absorbent material and the second impeding element. In yet other embodiments, the devices disclosed herein are configured to hold up to about 900 ml of fluid, up to about 600 ml of fluid or up to 300 ml of fluid. In still other embodiments, the devices disclosed herein further comprises an extension element extending the second fluid defining member to an area adjacent to or within the collection region of the collection vessel. In still other embodiments, the devices disclosed herein further comprise a cover. In still other embodiments, the first impeding element and the second impeding element are configured such that when the collection region of the collection vessel is filled with liquid to at least about 25% capacity by volume and sealed, in all orientations of the apparatus at least one of the first and second ends of the first fluid defining member is not submerged in liquid. In still other embodiments, the first impeding element and the second impeding element are configured such that when the collection region of the collection vessel is filled with fluid to at least about 50% capacity by volume and sealed, in all orientations of the apparatus at least one of the first and second ends of the first fluid defining member is not submerged in liquid. In some cases, the liquid is water. In some cases, the liquid is a physiological saline solution. As a non-limiting example, the physiological saline solution is defined in EN13726-1 as Test solution A.

Also included herein are methods for performing negative pressure wound therapy with the fluid collection apparatus devices disclosed herein. In some embodiments, the fluid collection apparatus devices disclosed herein comprises six sides, and the fluid collection apparatus in use is configured to be: (a) positioned with any of the six sides against a horizontal surface, and/or (b) hung from an attachment point on any of the six sides.

Also disclosed herein are methods of collecting fluid from a wound site of a subject, the method comprising: a) providing: i) a wound dressing positioned over the wound site, ii) a source of negative pressure, and iii) a fluid collection apparatus comprising a first fluid defining member in fluid communication with the source of negative pressure, a first impeding element positioned at a first end of the first fluid defining member, a second impeding element positioned at a second end of the first fluid defining member, and a second fluid defining member in fluid communication with the wound dressing; wherein the second fluid defining member defines a pathway for dispensing fluid drawn from the wound site into a fluid collection region of the fluid collection apparatus; and b) applying a negative pressure from the source of negative pressure to the wound site via the fluid collection apparatus to draw fluid from the wound site, through the second fluid defining member, and into the fluid collection region of the fluid collection apparatus; wherein the fluid comprises liquid and air, and fluid is retained in the fluid collection region and air is drawn through the first impeding element and/or the second impeding element, into the interior of first fluid defining member, and towards the source of negative pressure.

In some embodiments, the fluid collection apparatus of the methods disclosed herein comprises multiple sides, and the fluid collection apparatus in use is: (a) positionable with any of the sides against a horizontal surface and/or (b) hung from an attachment point on any of the sides. In some embodiments, the fluid collection apparatus is suspended from one or more attachment points on the fluid collection apparatus. In some embodiments, when the fluid collection region is full or nearly full of fluid and/or other material than air, and the fluid saturates the filter or the filter reaches a pre-defined saturation level, a pressure drop is generated or sensed to stop application of negative pressure. In some embodiments, up to about 900 ml of fluid, up to about 600 ml of fluid, or up to about 300 ml of fluid is retained in the fluid collection region.

Also disclosed herein are multi-orientation fluid collection apparatus devices for negative pressure wound therapy comprising: a) a collection vessel comprising a first side and a second opposing side, b) optionally, a cover connected to the first side of the collection vessel, c) a first fluid pathway in fluid communication with a source of negative pressure, the first fluid pathway positioned between: (i) a first impeding element positioned at an interior of the cover or interior of the first side of the collection vessel and a (ii) second impeding element positioned at an interior of the second side of the collection vessel, wherein the first impeding element and the cover or first side of the collection vessel are in substantially fluid tight communication, d) a filter positioned within the first fluid pathway and between the first impeding element and the second impeding element, and e) a second fluid pathway for introducing exudate into the collection vessel.

In some embodiments, the collection vessel of the devices disclosed herein comprises additional sides positioned between the first and second opposing sides, and the multi-orientation fluid collection apparatus in use is capable of being: (a) positioned with the first side, the second opposing side, or any of the additional sides against a horizontal surface, and/or (b) hung from an attachment point on the first side, the second opposing side, or any of the additional sides.

Also disclosed herein are multi-orientation fluid collection apparatus devices connected to a source of negative pressure for collecting wound exudate, the apparatus comprising a collection vessel; a first fluid pathway comprising a first and a second end with impeding elements at the first and second ends, said first fluid pathway and a first side of the apparatus in fluid tight or substantially fluid tight communication with the source of negative pressure; and a second fluid pathway for drawing fluid from a wound site into the apparatus. In some embodiments, in use said second fluid pathway and said wound site are in fluid tight or substantially fluid tight communication. In some embodiments, the devices comprise an air permeable member situated within the first fluid pathway. In some embodiments, the devices comprise a third fluid pathway situated within the first fluid pathway and in fluid communication with the air permeable member for receiving air from the air permeable member; said third fluid pathway within said first fluid pathway and a first side of the apparatus in fluid tight or substantially fluid tight communication and the third fluid pathway in fluid communication with the source of negative pressure. In some embodiments, the first fluid pathway is defined by a first fluid defining member. In some embodiments, the first fluid defining member is a chamber. In some embodiments, the second fluid defining member is a fluid inlet for receiving fluid from the wound site under negative pressure. In some embodiments, the air permeable member comprises one or more filters. In some embodiments, the filter comprises a hydrophobic filter. In some embodiments, the impeding elements comprise foam.

### BRIEF DESCRIPTION OF THE DRAWINGS

FIG. 1A depicts a front view of a first embodiment of a fluid collection apparatus.

FIG. 1B depicts a front view of a second embodiment of a fluid collection apparatus.

FIG. 2A depicts a cross-section view of the first embodiment of the fluid collection apparatus.

FIG. 2B depicts a cross-section view of the second embodiment of the fluid collection apparatus.

FIG. 3A depicts a detailed cross-section view of the first embodiment of the fluid collection apparatus.

FIG. 3B depicts a detailed cross-section view of the second embodiment of the fluid collection apparatus.

FIG. 4 depicts an exploded view of the first embodiment of the fluid collection apparatus.

FIG. 5 depicts a filter assembly of various embodiments of a fluid collection apparatus.

FIG. 6A depicts a bottom view of the first embodiment of the fluid collection apparatus.

FIG. 6B depicts a bottom view of the second embodiment of the fluid collection apparatus.

FIG. 7A depicts a front view of a third embodiment of a fluid collection apparatus.

FIG. 7B depicts a front view of a fourth embodiment of a fluid collection apparatus.

FIG. 8 depicts a perspective view of a fifth embodiment of a fluid collection apparatus.

FIG. 9 depicts a detailed cross-section view of the fifth embodiment of the fluid collection apparatus.

FIG. 10 is a photograph showing the front view of the fifth embodiment of the fluid collection apparatus.

FIG. 11 is a photograph showing the bottom view of the fifth embodiment of the fluid collection apparatus.

FIG. 12A shows the top, bottom, back, front, and left sides of the second embodiment of the fluid collection apparatus, as viewed from the left side of the apparatus.

FIG. 12B shows the top, bottom, left, right, and front sides of the second embodiment of the fluid collection apparatus, as viewed from the front side of the apparatus.

### DETAILED DESCRIPTION OF THE INVENTION

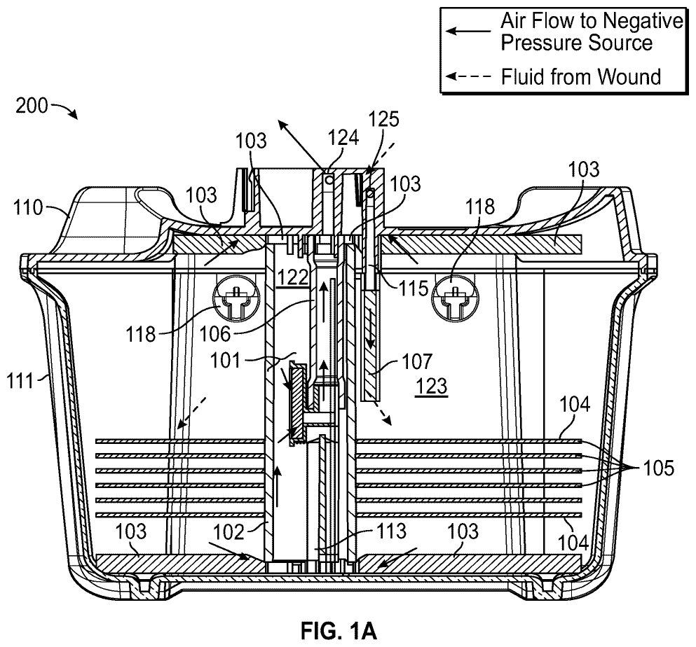

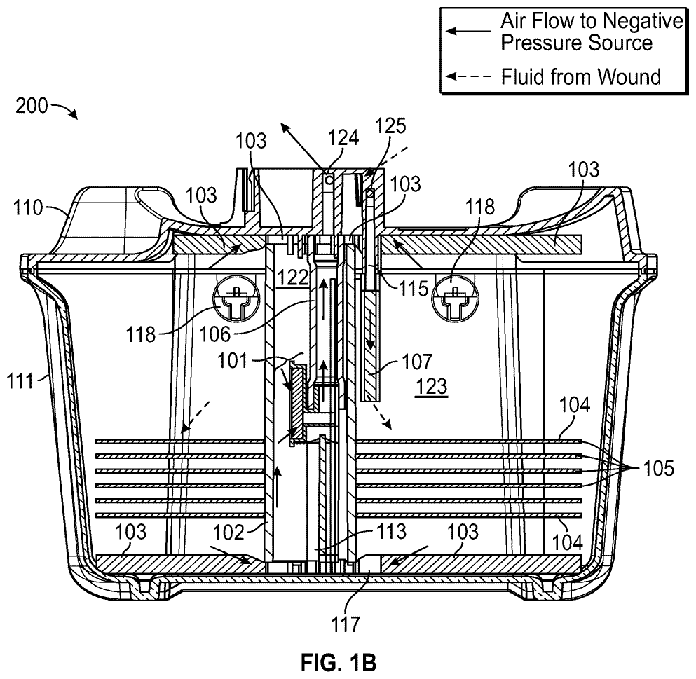

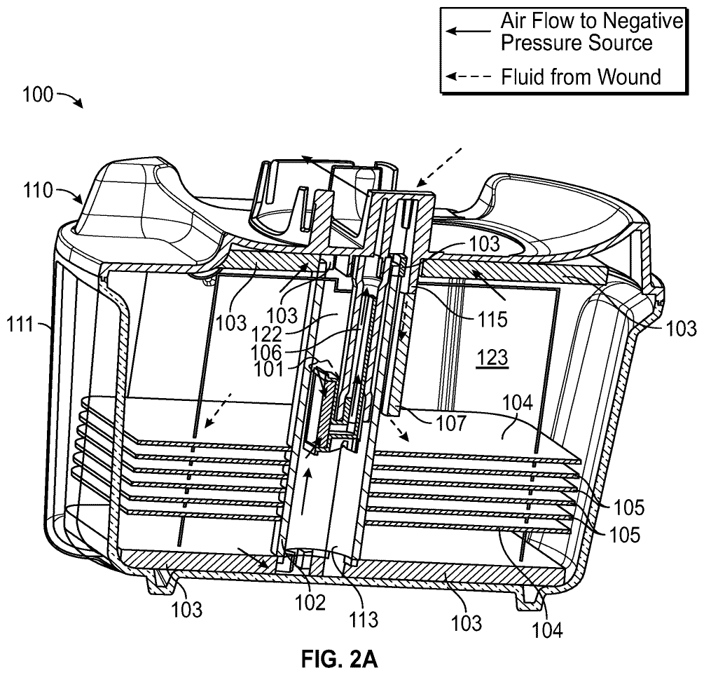

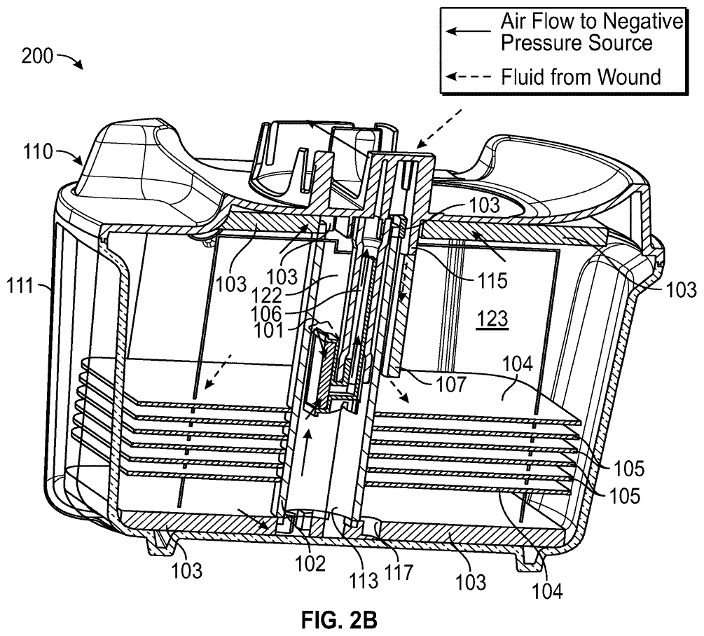

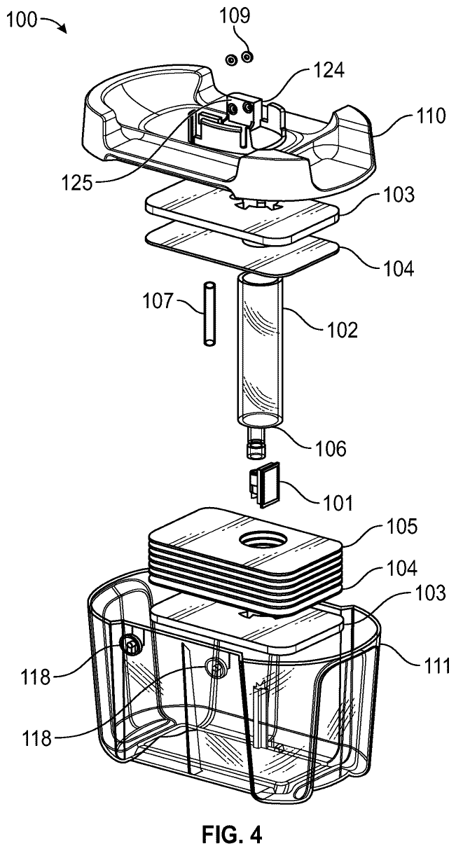

In one aspect, provided herein are fluid collection apparatuses for collecting fluid from a patient during negative pressure wound therapy (NPWT). A first exemplary embodiment 100 of a fluid collection apparatus is shown in a front side view in FIG. 1A and cross-section view in FIG. 2A. An exploded view of apparatus 100 is shown in FIG. 4. Apparatus 100 comprises a first fluid pathway, such as chamber 122, defined by a fluid defining member, such as chamber tube 102, configured to be in fluid communication with a source of negative pressure through a first pathway within the chamber. Apparatus 100 further comprises a second fluid pathway defined by a second fluid defining member, such as a fluid inlet tube 115, configured to be in fluid communication with a wound site such that during NPWT, negative pressure exerted on the wound site allows fluid, such as air and exudate, from the wound to travel to the apparatus. Fluid drawn from the wound site travels via the second fluid pathway to a collection region 123 of the apparatus where fluid can be stored, while air drawn from the wound site can be preferentially drawn into the chamber 122 and toward the source of negative pressure. In some embodiments, the pathway defining member 115 is a fluid inlet tube. In some embodiments, the second fluid defining member is part of a cover 110 of the apparatus. In some embodiments, negative pressure is applied to draw fluid from the wound via the following pathway: the source of negative pressure draws air through a first opening 124, from a fluid pathway within tube 106 and filter assembly 101 within chamber 122, and from impeding element 103 from the second fluid path way defined by the fluid defining member 115 (and extension tube 107, if present), such that fluid comprising air and liquid is drawn to the apparatus from the wound via 125. From the perspective of the negative pressure from the wound site to the source of negative pressure, the pathway would be the reverse (e.g., negative pressure draws air from the wound site through second fluid defining pathway 125 defined by second fluid defining member 115, through impeding element 103, to first fluid pathway 122 defined by first fluid defining member 102, through filter assembly 101 within the first fluid pathway 122, through a third fluid pathway defined by a third fluid defining member 106 and out of the first opening 124 to the source of negative pressure. Fluid flow into the apparatus from the wound site is shown in FIG. 1A by the hatched arrows. Air flow into the source of negative pressure is shown in FIG. 1B by the white arrows.

Apparatus 100 houses chamber 122 and collection region 123 within an interior region formed by a cover 110 and collection vessel 111. Cover 110 comprises a first opening 124 configured to connect the apparatus to a source of negative pressure and to provide a first pathway in fluid communication between the chamber of the apparatus and the source of negative pressure. Cover 110 further comprises a second opening 125 configured to connect the apparatus to a wound dressing and to provide a second pathway in fluid communication between the wound site and the apparatus. As shown in FIG. 4, a sealing member 109 is optionally provided at the first opening 124 and second opening 125 to connect with the source of negative pressure and wound dressing, respectively. In some cases, the sealing member 109 comprises an O-ring. In some cases, the sealing member comprises a flat gasket, a Luer fitting, conical fit, or push fit mechanism or the like. However, other features may be provided to establish and maintain fluid communication between the apparatus and the wound dressing, as well as the apparatus and source of negative pressure. For example, collection vessel 111 may further comprise a non-detachable top with couplings or connections between the collection vessel and wound dressing, as well as the collection vessel and source of negative pressure.

In an exemplary embodiment, air is preferentially drawn into the chamber 122 from the collection region 123 through a first impeding element 103 positioned at the top of the apparatus or a second impeding element 103 positioned at the bottom of the apparatus. Impeding elements 103 are shown in FIGS. 1-3. The first impeding element 103 is positioned at a first end of the chamber tube 102, and the second impeding element 103 is positioned at the second end of the chamber tube 102. In an exemplary embodiment, the first and second impeding elements are configured such that the pressure drop to draw liquid through the first and second impeding elements is higher than that to draw air through the first and second impeding elements. Accordingly, air may be preferentially drawn through the first or second impeding elements, through the chamber, and towards the source of negative pressure. The chamber tube 102 may be configured to hold the first and second impeding elements in place within the collection vessel 111. In some cases, the first impeding member may be pushed against cover 110 by the top of chamber tube 102 and the second impeding member may be pushed against the bottom of collection vessel 111 by the bottom of chamber tube 102, which compresses the first and second impeding members at the top and bottom sides of the chamber tube 102. In some cases, the impeding member may be compressed by about 100% to about 1000%. For instance, the impeding member is compressed from a thickness of about 3-10 mm to a thickness of about 0.5-5 mm. As a non-limiting example, the impeding member is compressed from a thickness of about 5 mm to a thickness of about 1 mm. For impeding members comprising a first uncompressed pore size, the compression of the first and/or second impeding members may reduce the uncompressed pore size by about 20% to about 80%. In some embodiments, the uncompressed pore size of the impeding element is from about 0.3 mm to about 0.5 mm. In some cases, the compressed pore size of the impeding element is from about 0.1 mm to about 0.2 mm. In some embodiments, the compressed pore size provides further preferential passage of air into the chamber as compared with liquid. The compressed pore size may also inhibit passage of particulates from wound fluid and/or absorbent material, if present, from entering into the chamber.

In some embodiments, the first and/or second impeding elements are configured to prevent the passage of particulates of about 100 microns to about 850 microns in size, e.g., about 400 microns in size. For a porous impeding element, the impeding element may comprise a tortuous path that hinders particulates smaller than the pore size from passing through the impeding element.

The impeding element may comprise an open-cell foam. In some cases, the foam comprises polyurethane, polyether, polyvinyl alcohol (PVA), or a combination thereof. For example, the impeding element comprises polyurethane foam. In some cases, the open-cell is compressed within the apparatus by the chamber tube 102, as described above. In some embodiments, the impeding element comprises a polypropylene, polyester, or rayon felt filter media such as supplied by Superior Felt and Filtration. In some cases, the impeding element comprises a polypropylene filter material, such as supplied by Pall Inc. In some embodiments, the impeding element comprises a fibrous material, for example, a polyester material. In some embodiments, the impeding element comprises a nonwoven material. Additional exemplary impeding elements are envisioned that comprise multiple small holes or capillaries through which air may preferentially pass during NPWT over liquid and/or particulate materials.

In some embodiments, the apparatus comprises an impeding element that is not positioned at either end of a chamber tube 102. As a non-limiting example, the impeding element is a series of small holes in a rigid structure e.g., radially holes or castellation slots through the wall of chamber tube 102 that may provide a fluid pathway that would provide resistance to liquid and preferential movement to air. In some such embodiments, chamber tube 102 may seal to the inside surface of the cover 110 and collection vessel 111. In some cases, the holes or slots are small enough to provide the appropriate flow resistance to liquid.

During NPWT, fluid is drawn into the collection region 123 through the pathway defining member 115 and optional fluid extension tube 107. Second fluid defining member 115 may extend to a desired length without the need for fluid extension tube 107, or may be connected to fluid extension tube 107 to establish an overall desired length of the two elements together. In a preferred embodiment, second fluid defining member 115 (with our without fluid extension tube 107) extends to a central region of collection region 123 to direct exudate and air to the central region. In some embodiments, the second fluid defining member 115 extends through the first impeding element. In other embodiments, the second fluid defining member 115 does not extend through the first impeding element. As a non-limiting example, the first impeding member may be formed such that wound fluid may pass into the collection region 123 without passing through the first impeding element. Collection region 123 may include an absorbent region comprising an absorbent material. Such absorbent material may be arranged as layers of absorbent material 105, and optionally wicking layers 104. If present, the wicking layers 104 are configured to transport liquid via capillary action through the wicking layers and into the layers of absorbent material to distribute fluid throughout the collection region 123. In some cases, the first and second impeding elements prevent the absorbent material from entering chamber 122.

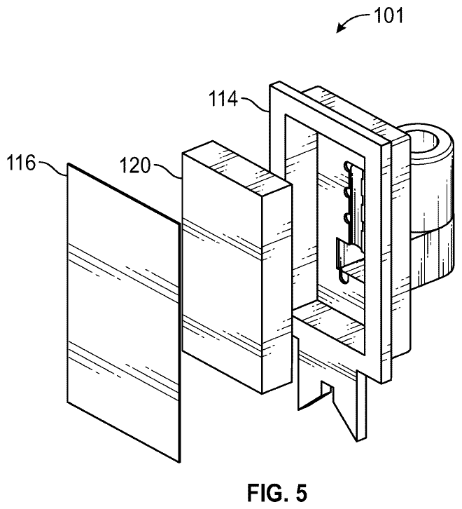

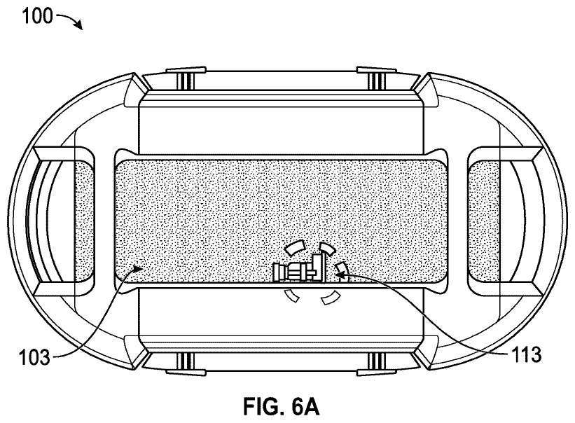

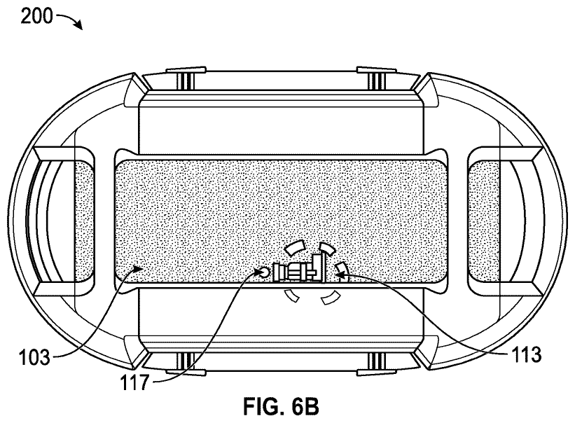

Apparatus 100 further comprises a filter assembly 101, which is shown in an exemplary embodiment in FIG. 5. For example, filter assembly 101 comprises a filter housing 114, an air permeable filter 116, for example a hydrophobic filter or other suitable filter that traps or prevents liquid passage, and optionally an aromatic filter 120, e.g., an activated carbon filter or other suitable aromatic filter. The filter assembly 101 is positioned within chamber 122 by, for example, filter tube 106. In some embodiments, the filter tube 106 is sealed against the cover 110 to create the first air pathway between the chamber of the apparatus and the source of negative pressure. In some embodiments, the filter tube 106 is moulded to the cover 110. In some embodiments, the filter tube 106 is connected to a spigot, hole, or other portion of the cover 110 by, e.g., a push-fit or other suitable mechanism. In some cases, the filter assembly 101 and 106 may be connected to or part of cover 110. In some embodiments, where apparatus 100 does not include a cover 110, filter tube 106 may be connected (directly or indirectly) to a side of the apparatus such that air under negative pressure may be drawn through filter tube 106 to the source of negative pressure through an opening in the apparatus. As air is drawn into the chamber 122 through the first and second impeding elements during NPWT, air may flow through the air permeable filter 116 and the aromatic filter 120, into filter tube 106, and toward the source of negative pressure through opening 124. Filter assembly 101 may be supported within chamber 122 by a support 113. FIG. 6A shows a bottom view of an exemplary apparatus 100 showing support 113 within chamber 122, surrounded by the second impeding element 103.

During NPWT, a negative pressure source and wound dressing are connected to the apparatus such that fluid (including air and liquid exudate) is drawn from the wound into the apparatus by exerted negative pressure. Wound fluid enters the collection region 123 through opening 125, passing through second fluid defining member 115 and optional extension tube 107, following a path as generally depicted by the hashed arrows in FIG. 1A. The fluid may then be drawn into absorbent layers 105, e.g., by the optional wicking layers 104 where the fluid is retained. Air enters the apparatus with fluid from the wound through second fluid defining member 115 and optional extension tube 107. Air may be further drawn through the path generally shown by the white arrows in FIG. 1A. Air drawn into the first impeding element and/or second impeding element 103 into chamber 122, can travel through filters 116 and 120, through filter tube 106, and out of the apparatus toward the negative pressure source through opening 124, while the wicking and/or absorbent material are configured to retain exudate in the collection region 123. Air permeable filter 116 may hinder liquid from exiting the apparatus through filter tube 106 and proceeding to the source of negative pressure. To restrict undesired materials, such as absorbent/wicking material and non-liquid exudate, from reaching filter 116, filter assembly 101 may be protected from direct contact with such material. In this example, chamber tube 102 surrounds filter assembly 101 to restrict such undesired material from reaching the filter 116.

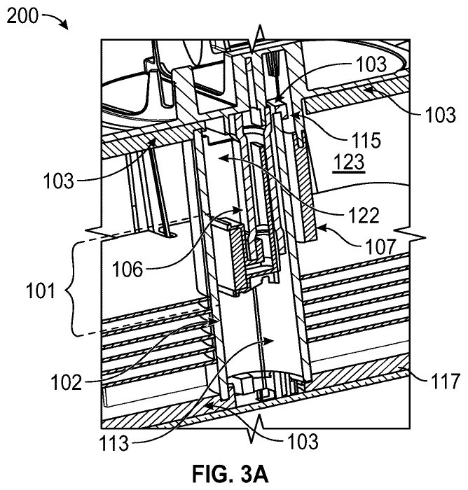

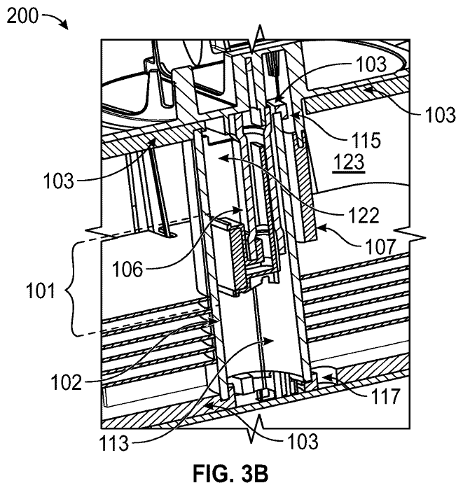

A second embodiment 200 of a fluid collection apparatus is shown from a front side in FIG. 1B and as a cross-section in FIG. 2B. FIG. 3B shows a detailed cross-section view of the second embodiment as shown in FIG. 2B. Apparatus 200 comprises the general features of apparatus 100, except the impeding element 103 positioned at the bottom of the apparatus comprises a hole 117 similar to the hole in the impeding element 103 positioned at the top of the apparatus through which second fluid defining member 115 is passed. FIG. 6B shows the bottom side of the apparatus 200, showing the hole 117 in the impeding element 103, while FIG. 6A shows the bottom of the apparatus 100, where the impeding element does not comprise the hole 117. The figures show first and second impeding elements 103, the filter tube 106, and the optional fluid extension tube 107. As shown in the figures, filter tube 106 may be connected to cover 110 to create the first fluid pathway in fluid connection with the negative pressure source. The features of FIG. 2B correspond to the features of FIG. 2A, except the embodiment shown in FIG. 2A is lacking a hole 117 in the second impeding element 103.

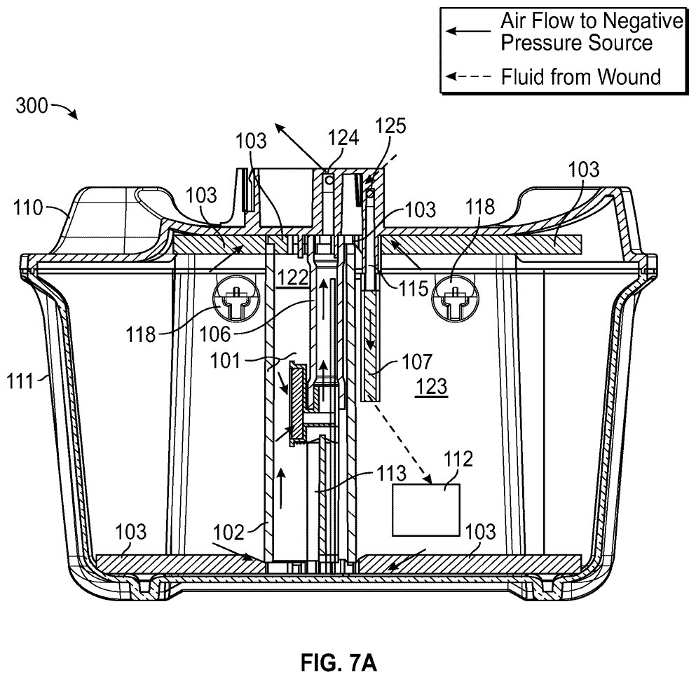

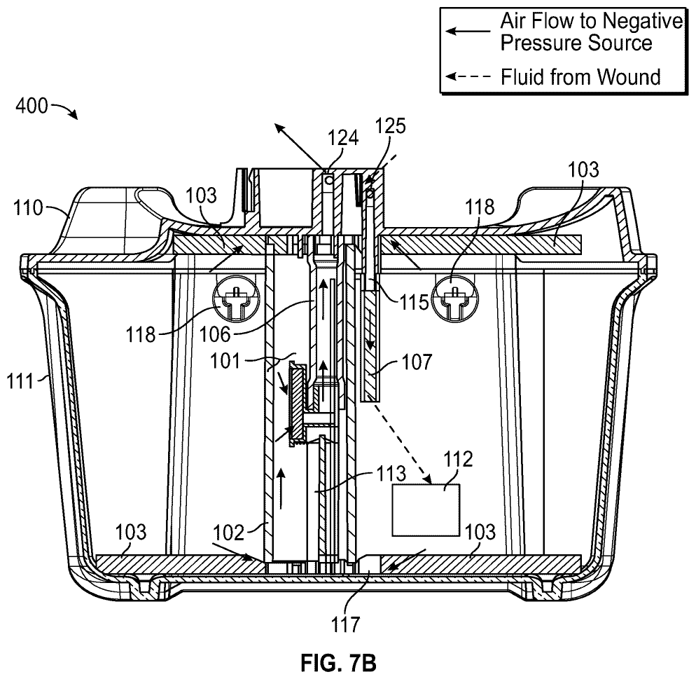

A third embodiment of a fluid collection apparatus is shown in FIG. 7A as apparatus 300. Apparatus 300 comprises the general features of apparatus 100, except the absorbent region of apparatus 300 may, for example, comprise an absorbent material within a bag, pouch, or other container, for example, a sachet 112, as opposed to layers of absorbent material. In some cases, the superabsorbent container, for example, a sachet, is dissolvable. While the drawing depicts an exemplary embodiment of a single sachet, the apparatuses and devices as disclosed herein may comprise a plurality of containers comprising superabsorbent, including at least one superabsorbent container, at least two superabsorbent containers, at least three superabsorbent containers, at least four superabsorbent containers, at least five superabsorbent containers or more, depending in part, for example, on the volume and dimensions of the apparatus.

In still other embodiments, the collection region 123 comprises an absorbent material, for example, superabsorbent material, such as superabsorbent granules, particles or other material capable of absorbing large amounts of liquid exudate relative to its own mass. Examples of superabsorbent polymers include, but are not limited to, cellulose or cellulose-derivative, for example, carboxymethyl cellulose, polyacrylate, including sodium polyacrylate, polyacrylamide and polyacrylamide co-polymers, ethylene maleic anhydride copolymers, crosslinked-carboxymethylcellulose, polyvinyl alcohol copolymers, cross-linked polyethylene oxide, polyacrylonitrile copolymers and combinations thereof. Absorbent material, for example superabsorbent polymers, may optionally include materials capable of absorbing odors or other noxious elements present in exudate and/or other excipients needed to, for example, increase particle size or surface area. In some cases, an odour absorbing material, such as activated carbon particles, may be included or added into to an impeding element, and/or wicking layer to provide an additional means to control odour such that air passing through the apparatus passes over odour absorbing material such as activated carbon.

A fourth embodiment of a fluid collection apparatus is shown in FIG. 7B as apparatus 400. Apparatus comprises the general features of apparatus 200, except the absorbent region of apparatus 400 comprises, for example, an absorbent material within a bag, pouch, or other container, for example, sachet 112.

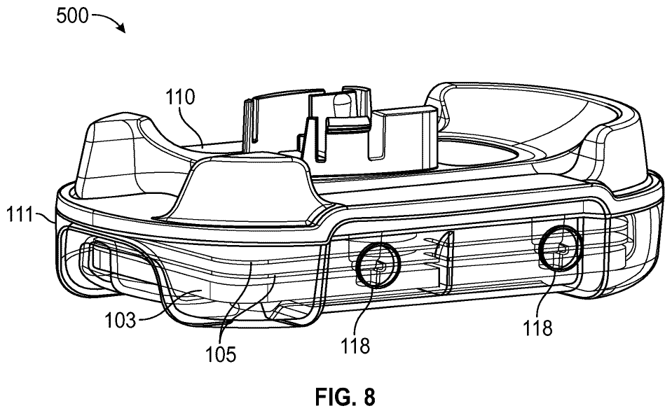

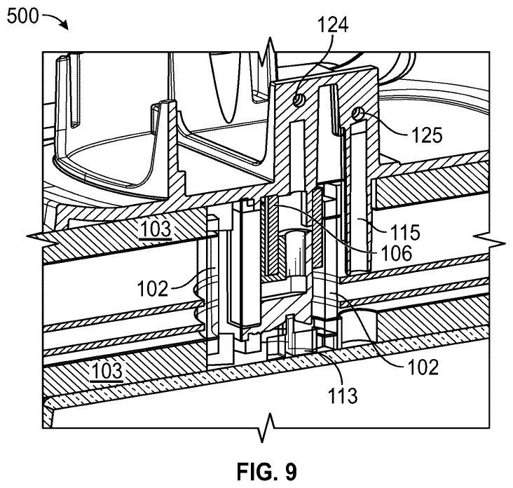

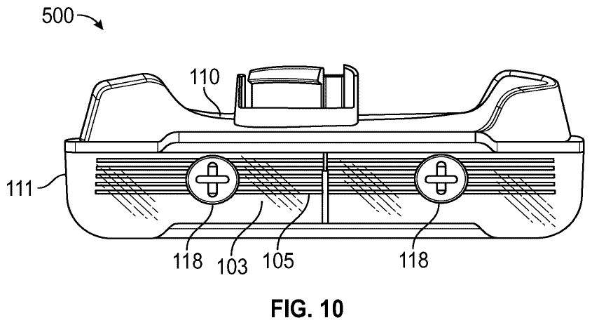

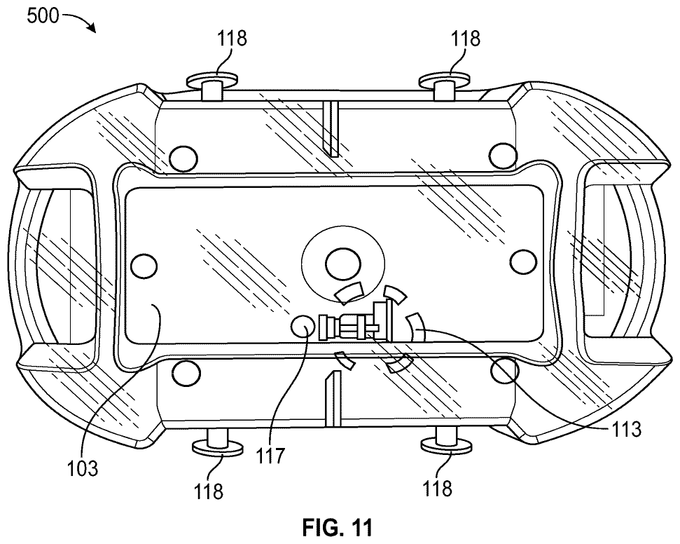

A fifth embodiment of a fluid collection apparatus is shown in FIGS. 8-11 as apparatus 500. Apparatus 500 is configured to store a smaller volume than each of apparatuses 100, 200, 300 and 400. For example, apparatuses and devices disclosed herein may be configured to store from about 100 ml to about 500 ml, from about 100 ml to about 200 ml, or from about 100 ml to about 300 ml of liquid. As a non-limiting example, apparatus 500 is configured to store about 300 ml of liquid. In comparison, larger apparatuses 100, 200, 300, and/or 400 may be configured to hold from about 300 ml to about 1200 ml, from about 400 ml to about 1000 ml, from about 400 ml to about 900 ml, from about 500 ml to about 1200 ml, from about 500 ml to about 100 ml, from about 500 ml to about 900 ml, from about 600 ml to about 1200 ml, from about 600 ml to about 100 ml, or from about 600 ml to about 900 ml of liquid. As non-limiting examples, the larger apparatuses 100, 200, 300 and/or 400 may each hold about 600 ml or 900 ml of liquid. The smaller apparatus 500 further differs from larger apparatuses 100, 200, 300, and 400 by lacking an extension tube 107, as shown in FIG. 9. In FIG. 9, the impeding elements 103, the second fluid defining member 115 and cover 110, and the filter tube 106 are shown.

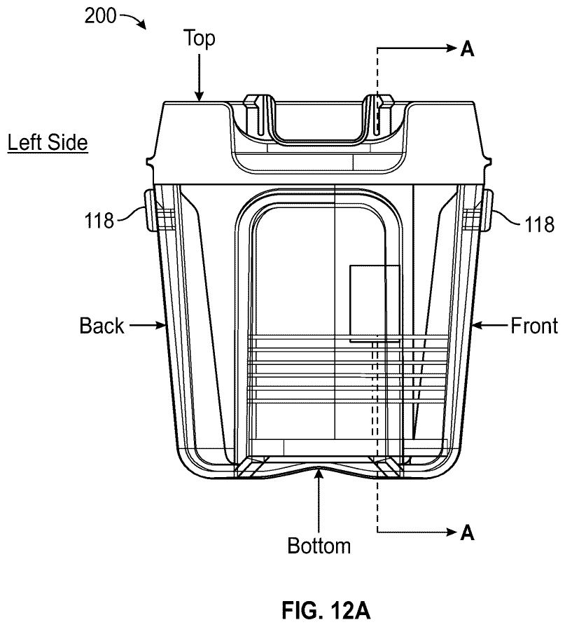

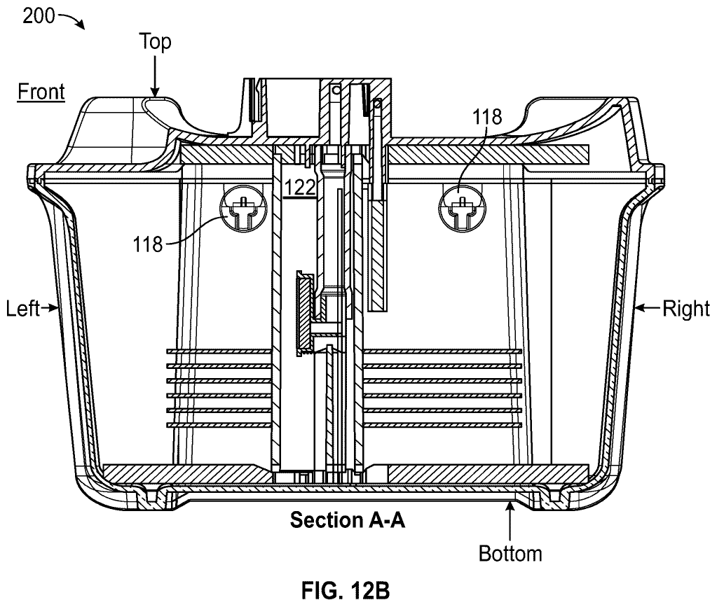

Each of the apparatuses shown may comprise attachment points 118 for hanging each apparatus in storage and/or during NPWT from one or more of its sides. The sides of apparatus 100 are depicted in FIGS. 12A and 12B. Apparatus 100 comprises six sides: a top side, a bottom side, a front side, a back side, a left side, and a right side. In some cases, the apparatus may be hung from a drip stand or other vertical pole by, for example, any of the six sides. In some cases, the apparatus may be positioned on a horizontal or substantially horizontal surface on any of the six sides. In some cases, the apparatus may be positioned such that the top of the cover 110 is facing downward, e.g., either resting at least partially against a horizontal surface or hanging. For the first, second, and fifth embodiments, the absorbent layers 105 may shift when the apparatus is positioned on its cover 110. For example, the absorbent layers may move adjacent to the cover and collect liquid in this orientation. For the third and fourth embodiments, absorbent material 112 positioned within the bag, sachet, pouch or other housing may also shift when the orientation of the apparatus is changed. For example, absorbent material 112 may be positioned adjacent to any of the six sides that are facing downward during use.

The apparatus and features thereof shown in FIGS. 1-12 are for illustrative purposes only and it is intended that a fluid collection apparatus may comprise additional components and/or lack one or more components shown. For example, an absorbent material and/or wicking layer may not be necessary for the fluid collection apparatus to function as described. As another example, an extension tube may be optional.

As used herein, a fluid is inclusive of a liquid and/or gas. As a non-limiting example, fluid drawn into an apparatus during a negative pressure therapy may comprise a mixture of liquid and gas, and the liquid may be retained within a collection region of the apparatus. In some cases, a fluid comprising a mixture of liquid and gas may be retained within the collection region. In some cases, the collection region comprises an absorbent material configured to absorb and retain liquid from a fluid drawn into the collection region, where the fluid drawn into the collection region comprises the liquid or a mixture of the liquid and a gas. In further cases, at least some of a gas drawn into the collection region may be retained within that region. In other cases, while there may be no net increase of gas in the collection region during a negative pressure therapy, there may instead be a net decrease of gas in the collection region during negative pressure therapy.

### Fluid Collection Apparatus

In one aspect, a fluid collection apparatus comprises a cover, a collection vessel, a chamber tube configured to be in fluid communication with a source of negative pressure, a first impeding element positioned at a first end of a first tube, a second impeding element positioned at a second end of the first tube, and a fluid inlet tube extending through the first impeding element. In some embodiments, the fluid inlet tube optionally comprises an extension such that the outlet end dispenses fluid drawn from a wound site of a patient during NPWT into a collection region of the collection vessel. In some instances, the first impeding element impedes fluid dispensed within the collection region from entering the first end of the first tube and the source of negative pressure. In some instances, the second impeding element impedes fluid dispensed within the collection region from entering the second end of the first tube and the source of negative pressure. The chamber tube may, for example, be configured to hold the first and the second impeding elements in place within the fluid collection apparatus. In some embodiments, the first and/or second impeding elements may comprise a material that allows air to flow freely within the material, while absorbing liquid exudate and/or restricting entry or movement of larger particles. In some embodiments, the impending element comprises foam, preferably an open cell foam. Exemplary foams include polyurethane, polyether, and polyvinyl alcohol (PVA). In some cases, the polyurethane foam is a reticulated polyurethane foam.

In one aspect, a fluid collection apparatus comprises a cover, a collection vessel, and an interior region comprising a chamber tube, a first impeding element positioned at a first end of the tube, a second impeding element positioned at a second end of the tube, and an absorbent material. In some instances, the absorbent material may be positioned external to the tube. In some embodiments, the first and second impeding elements may inhibit or prevent transfer of the absorbent material to the interior of the tube. In some embodiments, the first and/or second impeding elements may comprise foam; in other embodiments, the first and/or second impeding elements may comprise an open cell foam. Exemplary foams include polyurethane, polyether, and polyvinyl alcohol (PVA). In some cases, the polyurethane foam is a reticulated polyurethane foam.

In another aspect, a fluid collection apparatus comprises a cover, a collection vessel, a chamber tube, a first impeding element positioned at a first end of the tube, a second impeding element positioned at a second end of the tube, and a collection region external to the tube. In some embodiments, the first impeding element provides a first air path from the collection region to the interior of the tube, and the second impeding element provides a second air path from the collection region to the interior of the tube. In some embodiments, the first and/or second impeding elements may comprise foam; in other embodiments, the first and/or second impeding elements may comprise open cell foam. Exemplary foams include polyurethane, polyether, and polyvinyl alcohol (PVA). In some cases, the polyurethane foam is a reticulated polyurethane foam.

In another aspect, a fluid collection apparatus comprises a cover, a collection vessel, a fluid inlet tube, a fluid collection region, and at least one impeding element; wherein the fluid inlet tube optionally comprises an extension of an outlet end for dispensing fluid collected during negative pressure wound therapy into a fluid collection region of the collection vessel/In some embodiments, the fluid inlet tube extends through the impeding element such that the outlet end of the tube is positioned within the fluid collection region, and the outlet end of the tube is positioned adjacent to the absorbent region. In some instance, an optional extension may be provided to extend the outlet end of the fluid inlet tube such that the outlet end in larger apparatus configurations may be positioned adjacent to the absorbent region. In some embodiments, the first and/or second impeding elements may comprise foam; in other embodiments, the first and/or second impeding elements may comprise open cell foam. Exemplary foams include polyurethane, polyether, and polyvinyl alcohol (PVA). In some cases, the polyurethane foam is a reticulated polyurethane foam.

In one aspect, a fluid collection apparatus comprises a collection vessel, a fluid inlet tube, a fluid collection region, and at least one impeding element. In some instances, the fluid inlet tube optionally comprises an extension such that the outlet end of the fluid inlet tube is capable of dispensing fluid collected during negative pressure wound therapy into a fluid collection region of the collection vessel such that the outlet end of the fluid inlet tube may be positioned adjacent to or within the fluid collection region. In some instances, the outlet end of the fluid inlet tube may be positioned adjacent to an optional absorbent region. In yet other embodiments, the outlet end of the fluid inlet tube may be positioned within a region comprising, for example, superabsorbent material. In some instances, the superabsorbent may be contained within a bag, pouch or container, for example a sachet. In other instances, the bag, pouch or container may be dissolvable, for example, a dissolvable sachet. In some embodiments, the first and/or second impeding elements may comprise foam; in other embodiments, the first and/or second impeding elements may comprise open cell foam. Exemplary foams include polyurethane, polyether, and polyvinyl alcohol (PVA). In some cases, the polyurethane foam is a reticulated polyurethane foam.

In one aspect, a fluid collection apparatus comprises a collection vessel, a chamber tube configured to be in fluid communication with a source of negative pressure, a first impeding element positioned at a first end of a first tube, a second impeding element positioned at a second end of the first tube, and a fluid inlet tube extending through the first impeding element. In some embodiments, the fluid inlet tube optionally comprises an extension such that the outlet end dispenses fluid drawn from a wound site of a patient during NPWT into a collection region of the collection vessel. In some instances, the first impeding element impedes fluid dispensed within the collection region from entering the first end of the first tube and the source of negative pressure. In some instances, the second impeding element impedes fluid dispensed within the collection region from entering the second end of the first tube and the source of negative pressure. The chamber tube may, for example, be configured to hold the first and the second impeding elements in place within the fluid collection apparatus. In some embodiments, the first and/or second impeding elements may comprise a material that allows air to flow freely within the material, while absorbing liquid exudate and/or restricting entry or movement of larger particles. In some embodiments, the impending element comprises foam, preferably an open cell foam. Exemplary foams include polyurethane, polyether, and polyvinyl alcohol (PVA). In some cases, the polyurethane foam is a reticulated polyurethane foam. In some cases, the apparatus further comprises a cover configured to connect to the collection vessel.

In one aspect, a fluid collection apparatus comprises a collection vessel, and an interior region comprising a chamber tube, a first impeding element positioned at a first end of the tube, a second impeding element positioned at a second end of the tube, and an absorbent material. In some instances, the absorbent material may be positioned external to the tube. In some embodiments, the first and second impeding elements may inhibit or prevent transfer of the absorbent material to the interior of the tube. In some embodiments, the first and/or second impeding elements may comprise foam; in other embodiments, the first and/or second impeding elements may comprise an open cell foam. Exemplary foams include polyurethane, polyether, and polyvinyl alcohol (PVA). In some cases, the polyurethane foam is a reticulated polyurethane foam. In some cases, the apparatus further comprises a cover configured to connect to the collection vessel.

In another aspect, a fluid collection apparatus comprises a collection vessel, a chamber tube, a first impeding element positioned at a first end of the tube, a second impeding element positioned at a second end of the tube, and a collection region external to the tube. In some embodiments, the first impeding element provides a first air path from the collection region to the interior of the tube, and the second impeding element provides a second air path from the collection region to the interior of the tube. In some embodiments, the first and/or second impeding elements may comprise foam; in other embodiments, the first and/or second impeding elements may comprise open cell foam. Exemplary foams include polyurethane, polyether, and polyvinyl alcohol (PVA). In some cases, the polyurethane foam is a reticulated polyurethane foam. In some cases, the apparatus further comprises a cover configured to connect to the collection vessel.

In another aspect, a fluid collection apparatus comprises a collection vessel, a fluid inlet tube, a fluid collection region, and at least one impeding element; wherein the fluid inlet tube optionally comprises an extension of an outlet end for dispensing fluid collected during negative pressure wound therapy into a fluid collection region of the collection vessel/In some embodiments, the fluid inlet tube extends through the impeding element such that the outlet end of the tube is positioned within the fluid collection region, and the outlet end of the tube is positioned adjacent to the absorbent region. In some instance, an optional extension may be provided to extend the outlet end of the fluid inlet tube such that the outlet end in larger apparatus configurations may be positioned adjacent to the absorbent region. In some embodiments, the first and/or second impeding elements may comprise foam; in other embodiments, the first and/or second impeding elements may comprise open cell foam. Exemplary foams include polyurethane, polyether, and polyvinyl alcohol (PVA). In some cases, the foam is a reticulated polyurethane foam. In some cases, the apparatus further comprises a cover configured to connect to the collection vessel.

In another aspect, a fluid collection apparatus comprises a collection vessel; optionally, a cover connected to a first side of the collection vessel; a first fluid pathway in fluid communication with a source of negative pressure, the first fluid pathway positioned between: (i) a first impeding element positioned at an interior of the cover or interior of the first side of the collection vessel and a (ii) second impeding element positioned at an interior of a second side of the collection vessel opposing the first side, wherein the first impeding element and the cover or first side of the collection vessel are in substantially fluid tight communication; a filter positioned within the first fluid defining member and between the first impeding element and the second impeding element; and a second fluid pathway for introducing exudate into the collection vessel. In some embodiments, the first and second impeding elements serve as, for example, a pre-filter to inhibit liquid from reaching the filter positioned within the first fluid defining member. In some embodiments, the first and the second impeding elements are not hydrophobic filters. In some embodiments, the filter and the impeding elements do not comprise the same material. In some embodiments, the first and the second impeding elements preferentially allow passage of air to enter into the first fluid defining member, but as the collection vessel fills, some liquid may pass into the first fluid defining member. When sufficient liquid has entered into the first fluid defining member and covers the filter, a signal may be received indicating that the collection vessel could be full or at least about 50% full.

In some embodiments, any fluid collection apparatus described herein comprising an air permeable filter positioned within the chamber. In some cases, the air permeable filter comprises a hydrophobic filter. The air permeable filter may have a pore size of about 0.2 microns to 0.8 microns, or about 0.2 microns, 0.45 microns, or 0.8 microns. The air permeable filter may comprise polyethersulfone (PES), polytetrafluorethylene (PTFE) (e.g., as manufactured by Dow Corning), cellulose acetate, cellulose nitrate membranes, or a combination thereof. In some embodiments, the apparatus further comprises a carbon filter.

In some embodiments, any fluid collection apparatus described herein may comprise an absorbent material. The absorbent material may comprise a superabsorbent material. For example, the absorbent material may comprise a fibrous structure impregnated with the superabsorbent material. The superabsorbent material may, for example, also comprise cellulose or a cellulose-derivative. In some cases, the absorbent material comprises one or more layers of absorbent material. In some cases, the absorbent material may be provided within a sachet. The absorbent material may also be adjacent to a wicking layer.

In some embodiments any fluid collection apparatus described herein comprises six sides, and the fluid collection apparatus in use is configured to be: (a) positioned with any of the six sides against a horizontal surface, and/or (b) hung from an attachment point on any of the six sides.

### Fluid Collection Apparatus Components

In some embodiments, a fluid collection apparatus comprises a collection vessel and a cover. In some embodiments, the collection vessel may not include a separate cover but instead form all sides or walls of the vessel itself. The collection vessel may comprise a rigid plastic, e.g., a gamma sterilisable polycarbonate. An exemplary polycarbonate is Makrolon 2458. The cover may also comprise a rigid plastic such as a gamma sterilisable polycarbonate. Exemplary polycarbonates are Makrolon 2458 and RAL 9016. The cover may also comprise an O-ring for connecting the apparatus to a source of negative pressure and/or wound dressing. The O-ring may comprise nitrile, silicone, ethylene propylene diene monomer (EPDM), Viton, or a combination thereof. In some cases, the O-ring is black EPDM 70 Shore A.

The collection vessel may be configured to hold from about 100 ml to about 1200 ml of liquid in a collection region, e.g., a region comprising an absorbing material. In some cases, the collection vessel may hold about 100 ml, about 200 ml, about 300 ml, about 400 ml, about 500 ml, about 600 ml, about 700 ml, about 800 ml, about 900 ml, about 1000 ml, about 1100 ml, and about 1200 ml. In some embodiments, apparatus 100 is configured to hold about 600 ml of liquid. In some embodiments, apparatus 100 is configured to hold about 900 ml of liquid. In some embodiments, apparatus 200 is configured to hold about 600 ml of liquid. In some embodiments, apparatus 200 is configured to hold about 900 ml of liquid. In some embodiments, apparatus 300 is configured to hold about 300 ml of liquid.

In some embodiments, the first and second impeding elements of the apparatus comprise foam. The foam may be in the form of a layer that provides an air path from the collection region of the canister to the chamber and on to the negative pressure source. The foam may also prevent transfer of absorbing material to the filter assembly. In some cases, the foam is an open cell foam. In some cases, foam comprises polyurethane, polyether, polyvinyl alcohol (PVA), or a combination thereof. In an exemplary embodiment, the foam comprises polyurethane, e.g., a reticulated polyurethane foam. An exemplary reticulated polyurethane foam is Blue RAL 5017.

In some embodiments, a fluid collection apparatus comprises an absorbent region comprising an absorbent material. In some cases, the absorbent material comprises one or more layers of absorbent material. In some cases, the absorbent material has about 1, 2, 3, 4, 5, 6, 7, 8, 9, or 10 layers of absorbent material. As an exemplary embodiment, the absorbent material has 6 layers of absorbent material. In some cases, the absorbent material is provided within a sachet. The sachet may be dissolvable.

In some aspects, an absorbent material comprises a super absorbent material. Non-limiting examples of super absorbent materials include a material or combination of materials that absorb about or at least about 10-, 20-, 30-, 40-, 50-, 60-, 70-, 80-, 90-, 100-, 120-, 140-, 160-, 180-, 200-, 250-, 300-, 400-, or 500-times the super absorbent material's weight in water. In some cases, a super absorbent material absorbs about 20-500 times its weight in water, or absorbs about 50-500 times its weight in water. When the super absorbent is used in a bag or sachet for retaining biological fluids having salinity such as exudates, the super absorbent fluid may absorb between about 4 and about 10 times its weight in a saline liquid.

In some aspects, an absorbent material expands from a first thickness to a second thickness upon absorption of fluid, wherein the second thickness is less than or equal to the maximum thickness of the absorbent material. In some embodiments, the first thickness refers to the thickness of the absorbent material prior to absorption of fluid during a negative pressure therapy. For example, the first thickness is the thickness of the expandable absorbent material supplied and/or stored with a fluid collection apparatus for use in negative pressure therapy. In some embodiments, the absorbent material is a super absorbent material that expands during absorption of fluid. In some cases, the first thickness of the expandable absorbent material is between about 3 mm and 15 mm, or between about 5 mm and 10 mm. In some cases, the maximum thickness is between about 15 mm and 50 mm, or between about 20 mm and 35 mm. In some cases, the maximum thickness of the expandable absorbent material is about 1.2-, 1.4-, 1.6-, 1.8-, 2-, 2.5-, 3-, 3.5-, 4-, 4.5- or 5-times the first thickness. In some cases, the maximum thickness of the expandable absorbent material is about 1.5-5, 1.5-4, 1.5-3, 1.5-2.5, or 1.5-2 times the first thickness.

Non-limiting examples of absorbent materials include polyacrylate, non-woven material, cellulose fibres, tissue paper, polyacrylamide copolymer, and combinations thereof. A non-woven material includes a polyester staple fibre. In a non-limiting example, an apparatus comprises the superabsorbent polymer polyacrylate. As another non-limiting example, an apparatus comprises the superabsorbent polymer Needlefelt type 0570N700400 (Technical Absorbents). In some cases, an apparatus comprises two or more materials with absorbing properties. In some cases, an apparatus comprises a mixture of super absorbent polymer and cellulose fibers. In some embodiments, an absorbent material comprises a base fibrous structure impregnated with super absorbing polymer particles. For example, Specificall cellulose Absorbent Pad 113.

In some embodiments, an absorbent material is in a powder or granular form within a bag or sachet. In some embodiments, the absorbent material is enclosed within a casing within the apparatus. In some embodiments, the absorbent material comprises a superabsorbent polymer. The casing is sometimes referred to as a transmissive material or layer which allows fluid to flow into the casing to the absorbent material, while retaining the absorbent material within the casing. In some cases, the transmissive material has a wicking property, where fluid transfer into the casing is facilitated by the wicking property of the transmissive material, for example, via capillary action. In other or additional cases, a separate and/or additional wicking layer is provided on an exterior of the casing to draw liquid into the casing to the absorbing material. In some cases, a layer of the transmissive material enclosing the absorbent material is between about 0.02 mm and 0.2 mm thick or between about 0.08 mm and 0.15 mm thick. Non-limiting examples of transmissive materials include non-woven polypropylene, cellulose fibres, non-woven HDPE and a combination thereof.

In some embodiments, an absorbent material is provided in the collection region of the apparatus as loose particles, e.g., superabsorbent particles that are not contained in a bag or casing and are not formed as layers.

In some aspects of the disclosure, a fluid collection apparatus comprises a wicking material. Wicking materials include materials configured to receive liquid and then rapidly transport the liquid, for example, via capillary action, to another material adjacent the wicking material. For instance, the wicking material receives liquid drawn into the collection region of the apparatus and then transfers the liquid to the absorbent material, where the absorbent material absorbs and retains the liquid. In some embodiments, a wicking material wicks more than 15 mm of water vertically over a time period of 24 hours. In some cases, the absorbent material is a superabsorbent polymer. In exemplary embodiments, the apparatus comprises a wicking material positioned adjacent an absorbent material described herein. Non-limiting examples of wicking materials include cellulose pulp, cotton, tissue paper, non-woven polyester, and a combination thereof. In some configurations, about a 0.05-10 mm, or about a 0.2-2 mm layer of wicking material is positioned adjacent to an absorbent material.

For an apparatus comprising an absorbent region comprising an absorbent material and optionally a wicking material, the distance between the fluid inlet tube or extension tube and the absorbent region may be from about 10 mm to about 60 mm. As a non-limiting example, for an apparatus configured to hold about 300 ml of liquid, the distance is about 15 mm. As another example, for an apparatus configured to hold about 600 ml of liquid, the distance is about 30 mm. As a further example, for an apparatus configured to hold about 900 ml of liquid, the distance is about 40 mm.

The first fluid defining member, or chamber tube, that provides space for the filter assembly may be rigid. This member may be cut from an extruded tube or moulded to suit a particular apparatus. As a non-limiting example, a first fluid defining member comprises clear polycarbonate, such as Makrolon 2458. In some embodiments, the first fluid defining member has a circular, square, rectangular, or other cross section. For an apparatus comprising a circular cross section, the filter assembly may be positioned within the chamber such that the face of the filter is held away from a wall to allow for free passage of air. In some cases, the inner diameter of the chamber tube is from about 10 mm to about 24 mm, from about 12 mm to about 24 mm, from about 14 mm to about 24 mm, from about 16 mm to about 24 mm, from about 18 mm to about 24 mm, from about 12 mm to about 22 mm, from about 12 mm to about 20 mm, or from about 12 mm to about 18 mm. As a non-limiting example, the inner diameter is about 18 mm. In some cases, the outer diameter of the chamber tube is from about 16 mm to about 28 mm, from about 18 mm to about 28 mm, from about 20 mm to about 28 mm, from about 22 mm to about 28 mm, from about 16 mm to about 26 mm, from about 16 mm to about 24 mm, or from about 16 mm to about 22 mm. As a non-limiting example, the outer diameter is about 22 mm. In some embodiments, the first fluid defining member may be permanently affixed or part of the cover or collection vessel.

The filter assembly may comprise an air permeable filter, an aromatic filter, and a filter housing. As a non-limiting example, the air permeable filter comprises a hydrophobic filter. In some cases, the filter housing is moulded in Natural ABS, e.g., as provided by Novodur FID M203FC. In some cases, the aromatic filter comprises carbon for reducing odors during NPWT. In some cases, the aromatic filter comprises from about 25 g/m2 to about 200 g/m2, from about 25 g/m2 to about 175 g/m2, from about 25 g/m2 to about 150 g/m2, from about 25 g/m2 to about 125 g/m2, from about 25 g/m2 to about 100 g/m2, from about 50 g/m2 to about 200 g/m2, or from about 75 g/m2 to about 200 g/m2 activated carbon. For example, the aromatic filter comprises about 80 g/m2, about 90 g/m2, or about 100 g/m2 activated carbon. An exemplary aromatic filter comprises activated carbon and a non-woven material with an enhanced binder. For instance, the non-woven blend comprises polyester, polyolefin, and activated carbon. As a non-limiting example, the non-woven blend comprises about 88% polyester and about 12% polyolefin, with about 90 g/m2 activated carbon (e.g., as provided by Sterling non-wovens, material number 3351).

In some embodiments, the air permeable filter has a pore size from about 0.2 microns and about 0.8 microns, or about 0.2 microns, about 0.45 microns, or about 0.8 microns. A first exemplary air permeable filter comprises polyethersulfone (PES). For example, a PES filter membrane on polyester nonwoven support, e.g., as provided by Pall, having material number S80535 (0.45 micron pore size, 0.76 mm to 0.162 mm thickness). The air permeable filter may alternatively or additional comprises polytetrafluorethylene (PTFE), cellulose acetate, cellulose nitrate membranes, or a combination thereof.

In some embodiments, the filter tube is elastomeric. In some embodiments, the filter tube is plastic. As non-limiting examples, the filter tube comprises gamma sterilisable materials such as polyvinyl chloride (PVC), silicone, ethylene propylene diene monomer (EPDM), Viton. In some cases, the filter tube comprises PVC 60 Shore A.

The apparatus may comprise a fluid inlet tube, and optionally, an extension tube. The extension tube, and/or fluid inlet tube, may comprise gamma sterilisable PVC, silicone, EPDM, Vitron, or a combination thereof. For example, the extension tube comprises PVC 60 Shore A. In some cases the extension tube attaches to the apparatus cover. In some cases, the extension tube is included in the cover moulding.

### Systems and Kits

In one aspect of the disclosure, provided herein are systems comprising a fluid collection apparatus described herein and one or more accessory elements. Accessory elements include materials useful for performing a negative pressure therapy such as NPWT. In some embodiments, an accessory comprises a wound dressing. A wound dressing includes, without limitation, a dressing having a cover for sealing around a wound site and maintaining a negative pressure environment at the wound site, where the cover further comprises an adhesive for the sealing and an opening for the transfer of negative pressure. Non-limiting examples of wound dressing covers include polyurethane films having, for example, a polyurethane adhesive. In some embodiments, an accessory comprises a source of negative pressure. In some embodiments, “negative pressure” refers to pressure below atmospheric pressure. Sources of negative pressure include pumps configured to maintain a negative pressure between about 60 mmHg and about 145 mmHg below atmospheric pressure. For example, the pump may be configured to maintain a negative pressure of about 80, 100 or 125 mmHg below atmospheric pressure, +20 mmHg. Sources of negative pressure include pumps configured to exert a maximum negative pressure of at least 200 mmHg, at least 330 mmHg or at least 400 mmHg. In exemplary embodiments, a pump is a diaphragm pump. In exemplary embodiments, a pump may be an electric pump, either mains or battery powered. The pump may be configured to operate continuously. In further exemplary embodiments, the pump may be a medical pump, such as a pump complying with Directive 93/42/EEC: IIA, IEC 60601-1 and/or IEC 60601-1-2. Additional accessory items include one or more conduits or tubings configured to connect the fluid collection apparatus to a source of negative pressure and/or wound dressing; and a connector configured to connect the outlet of the apparatus to a source of negative pressure, and connect the inlet of the apparatus to the wound dressing.

### Methods

Provided herein are methods for collecting fluid using a fluid collection apparatus described herein. In some embodiments, the fluid collection apparatus utilized comprises a first fluid defining member, such as a chamber, in fluid communication with a source of negative pressure, a first impeding element positioned at a first end of the first fluid defining member, a second impeding element positioned at a second end of first fluid defining member, and a second fluid defining member, such as a fluid inlet tube, in fluid communication with a wound dressing; wherein the fluid inlet tube comprises an outlet end and the fluid inlet tube extends through the first impeding element such that the outlet of the fluid inlet tube is positioned within a fluid collection region of the fluid collection apparatus.

In one aspect, the method comprises applying a negative pressure from the source of negative pressure to the wound site via the fluid collection apparatus to draw fluid from the wound site, through the fluid inlet tube, and into the fluid collection region of the fluid collection apparatus; wherein the fluid comprises liquid and air, and the fluid is retained in the fluid collection region and the air is drawn through the first and/or the second impeding elements, into the interior of chamber tube, and towards the source of negative pressure. In some embodiments, the negative pressure applied from the negative pressure source is between about 75 mmHg and about 125 mmHg below atmospheric pressure.

In some methods, the fluid collection apparatus is positioned in an orientation-independent manner. In some cases, the fluid collection apparatus comprises multiple sides (e.g., six sides), and the fluid collection apparatus in use is: (a) positioned with any of the multiple sides against a horizontal surface and/or (b) hung or secured from an attachment point on any of the multiple sides. In some cases, the fluid collection apparatus is suspended, attached or otherwise secured from one or more attachment points on the fluid collection apparatus. In some cases, the orientation independence of an apparatus described herein is evaluated by filling the collection vessel with a liquid. In some cases, the liquid is water. In some cases, the liquid is a physiological saline solution. As a non-limiting example, the physiological saline solution is defined in EN13726-1 as Test solution A.

### EXAMPLES

### Example 1: Fluid Collection Apparatus

A fluid collection apparatus as generally depicted in FIG. 1 was manufactured. The components, canister header (110) and canister base (111) were injection moulded from suitable material. The chamber tube (102) was cut from extruded tube having a rigid material such as poly carbonate, or manufactured by injection moulding as a discrete component. The filter tube (106) and extension tube (107) were cut from a flexible tube material comprising PVC that had previously been extruded to shape. The filter assembly (101) was manufactured from three components: an outer housing (114), which was injection moulded from a plastic suitable for ultrasonic welding or thermally welding to the hydrophobic filter material (116), an activated carbon filter element (120) that is contained by the filter housing (114), and a hydrophobic filter (116) which is welded to the housing (114) to seal to the edges and retain the carbon filter.

The absorbent layers (105), foam (103) and wicking sheet (104) components were stamped from roll stock. The absorbent layers comprise superabsorbent polymers. A foam sheet (103), wicking sheet (104) and the absorbent layers (105) were loaded into the canister base (111) and the filter tube (106) was placed into the canister base so that it was located by alignment features in the base of the canister and trapped the foam sheet (103) and wicking sheet (104) against the base of the canister. The absorbent layers (105) were free to slide over the chamber tube (102). A foam sheet (103) and wicking sheet (104) were fitted to the canister header (110). The filter tube (106) was pressed onto the tube spigot of the filter housing of the filter sub assembly (101) and the opposite end of the filter tube (106) was pressed over the tube spigot of the header (110) so that the filter assembly (101) is connected to the header. Optionally the canister filling tube is fitted to the fluid inlet spigot on the canister header (110). The two partial assemblies (upper and lower) were brought together so that the filter assembly (101) fits within the canister tube (102) and the mating surfaces of the canister are brought together. In this example, the mating surfaces of the canister header (110) and canister base (111) have features to aid joining them together. In the case of ultrasonic welding being employed for the joining process, a weld concentrating feature is designed into one component and a receiving surface in the other. This feature pairing may be mating pair of a tongue and groove features on the mating surfaces with a small welding concentrator such as a small radius bump (0.2 to 1 mm diameter) on the end of the tongue feature to concentrate the welding energy. The assembly is typically placed in an ultrasonic welding feature and the two halves are welded together by application of ultrasonic energy to one component to forma seal with the other part. Optionally additional sealing can be provided by solvent or adhesive should this be necessary.

### Example 2: Orientation Independence of a Fluid Collection Apparatus

To test the orientation independence of the apparatus, liquid representative of wound exudate was supplied to the apparatus at rate representative of anticipated use. This test was continued until the canister reached capacity and the hydrophobic filter was obscured by liquid. This test has been conducted with the apparatus in a typical orientation, with the canister resting on its base. This test has further been conducted with the canister placed on its side and on one end of the apparatus. The most challenging orientation is with the long axis at or close to vertical as this requires fluid to travel a greater vertical distance against gravity. However, the wicking layer assists performance under these conditions, ensuring that the majority of the absorbent layers were saturated before liquid reached the hydrophobic filter within the chamber tube.

While preferred embodiments of the present disclosure have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the inventions described herein may be employed in practicing the inventions. It is intended that the following claims define a scope of the inventions and that methods and structures within the scope of these claims and their equivalents be covered thereby.

1-40. (canceled)

41. A method of collecting fluid from a wound site of a subject, the method comprising:
a) providing:
i) a wound dressing positioned over the wound site,
ii) a source of negative pressure, and
iii) a fluid collection apparatus comprising a first fluid defining member in fluid communication with the source of negative pressure, a first porous impeding element positioned at a first end of the first fluid defining member, a second porous impeding element positioned at a second end of the first fluid defining member, and a second fluid defining member in fluid communication with the wound dressing;
wherein the second fluid defining member defines a pathway for dispensing fluid drawn from the wound site into a fluid collection region of the fluid collection apparatus; and
b) applying a negative pressure from the source of negative pressure to the wound site via the fluid collection apparatus to draw fluid from the wound site, through the second fluid defining member, and into the fluid collection region of the fluid collection apparatus;
wherein the fluid comprises liquid and air, and fluid is retained in the fluid collection region and air is drawn through the first porous impeding element and/or the second porous impeding element, into the interior of first fluid defining member, and towards the source of negative pressure.

42. (canceled)

43. (canceled)

44. The method of claim 41, wherein the fluid collection apparatus further comprises a filter positioned within the first fluid defining member.

45. (canceled)

46. The method of claim 44, wherein the filter comprises a pore size of between 0.2 micron and 0.8 micron.

47. The method of claim 44, wherein the filter comprises polyethersulfone (PES), polytetrafluorethylene (PTFE), cellulose acetate, cellulose nitrate membranes, or a combination thereof.

48. The method of claim 47, wherein the filter impedes liquids and solids from reaching the source of negative pressure.

49-55. (canceled)

56. The method of claim 41, wherein at least one of the first porous impeding element or the second porous impeding element comprises reticulated polyurethane foam.

57-71. (canceled)

72. The method of claim 41, wherein the second fluid defining member extends through the first impeding element and comprises an outlet end positioned adjacent to a first wicking layer such that fluid drawn into the fluid collection apparatus is capable of being drawn into the absorbent material via the first wicking layer.

73-102. (canceled)

103. A multi-orientation fluid collection apparatus for negative pressure wound therapy comprising:
a) a collection vessel comprising a first side and a second side,
b) a cover connected to the first side of the collection vessel,
c) a first fluid pathway in fluid communication with a source of negative pressure, the first fluid pathway positioned between:
(i) a first porous impeding element positioned at an interior of the cover or interior of the first side of the collection vessel, and
(ii) a second porous impeding element positioned at an interior of the second side of the collection vessel,
wherein the first porous impeding element is in fluid tight communication with one of the cover or the first side of the collection vessel, and
d) a second fluid pathway for introducing exudate into the collection vessel.

104. The multi-orientation fluid collection apparatus of claim 103 wherein the apparatus is orientation independent.

105. The multi-orientation fluid collection apparatus of claim 103, wherein the second porous impeding element is in fluid tight communication with the second side of the collection vessel.

106. The multi-orientation fluid collection apparatus claim 103, further comprising a manifold, wherein a first inlet of the manifold is fluidly connected to the first porous impeding element, wherein a second inlet of the manifold is fluidly connected to the second porous impeding element, and wherein an outlet of the manifold is fluidly connected to the source of negative pressure.

107. The multi-orientation fluid collection apparatus of claim 106, wherein the manifold further comprises a filter positioned within the first fluid pathway between the source of negative pressure and the first and second porous impeding elements.

108. (canceled)

109. The multi-orientation fluid collection apparatus of claim 107, wherein the filter comprises a pore size of between 0.2 micron and 0.8 micron.

110. The multi-orientation fluid collection apparatus of claim 107, wherein the filter comprises polyethersulfone (PES), polytetrafluorethylene (PTFE), cellulose acetate, cellulose nitrate membranes, or a combination thereof.

111. (canceled)

112. (canceled)

113. The multi-orientation fluid collection apparatus of claim 103, wherein the first porous impeding element and/or the second porous impeding element is/are configured to impede flow of liquid and solids therethrough, but to enable flow of gas therethrough.

114-118. (canceled)

119. The multi-orientation fluid collection apparatus of claim 103, further comprising an absorbent material.

120. (canceled)

121. The multi-orientation fluid collection apparatus of claim 119, wherein the absorbent material comprises a fibrous structure impregnated with superabsorbent material.

122. (canceled)

123. (canceled)

124. The multi-orientation fluid collection apparatus of claim 119, wherein the absorbent material is provided within a dissolvable sachet.

125. (canceled)

126. (canceled)

127. The multi-orientation fluid collection apparatus of claim 119, further comprising a first wicking layer positioned between the first porous impeding element and the absorbent material.

128. (canceled)

129. The multi-orientation fluid collection apparatus of claim 127, further comprising a second wicking layer positioned between the absorbent material and the second porous impeding element.

130-139. (canceled)
